(19) United States
(12) Patent Application Publication
(10) Pub. No.: US 2005/0257329 A1
(43) Pub. Date: Nov. 24, 2005

(54) Composition for dyeing keratin fibers, comprising at least one alcohol oxidase and at least one cationic oxidation base, and process using this composition

(76) Inventor: Gregory Plos, Tokyo (JP)

Correspondence Address: Thomas L. Irving;FINNEGAN, HENDERSON, FARABOW,, GARRETT & DUNNER, L.L.P., 901 New York Avenue, N.W., Washington, DC, 20001-4413, US

(21) Appl. No.: 11/044,080

(22) Filed: Jan. 28, 2005

Related U.S. Application Data
(60) Provisional application No. 60/544,324, filed on Feb. 17, 2004.

(30) Foreign Application Priority Data
Jan. 28, 2004 (FR) 0400775

(51) Int. Cl. A61K 7/13

(52) U.S. Cl. 8/405

(57) ABSTRACT

Disclosed herein is a composition for dyeing keratin fibers, for example, human keratin fibers such as the hair, comprising, in a medium suitable for dyeing, at least one alcohol oxidase enzyme, at least one substrate for the enzyme, and at least one cationic oxidation base. Further disclosed herein are a process for dyeing keratin fibers, comprising applying this composition to the keratin fibers, and a dyeing “kit”.

This application claims benefit of U.S. Provisional Application No. 60/544,324, filed Feb. 17, 2004.

The present disclosure relates to a composition for dyeing keratin fibers, for example, human keratin fibers such as the hair, comprising, in a medium suitable for dyeing, at least one cationic oxidation base, at least one alcohol oxidase enzyme, and at least one substrate for the enzyme.

It is known practice to dye keratin fibers, such as human hair, with dye compositions comprising oxidation dye precursors, such as ortho- or para-phenylenediamines, ortho- or para-aminophenols, and heterocyclic compounds, which are generally referred to as oxidation bases. These oxidation bases are colorless or weakly colored compounds which, when combined with oxidizing products, may give rise to colored compounds by a process of oxidative condensation.

It is also known that the shades obtained with these oxidation bases may be varied by combining them with couplers or coloration modifiers, which may be chosen, for example, from aromatic meta-diamines, meta-aminophenols, meta-diphenols and certain heterocyclic compounds such as indole compounds.

The variety of molecules used as oxidation bases and couplers makes it possible to obtain a wide range of colors.

The “permanent” coloration obtained by these oxidation dyes should satisfy a certain number of requirements. For example, it should have no toxicological drawbacks and it should allow shades of the desired intensity to be obtained and have good resistance to external agents, such as light, bad weather, washing, permanent waving, perspiration and rubbing.

The dyes should also allow white hairs to be covered and they should be as unselective as possible. For example, they should allow the smallest possible differences in coloration to be produced over the entire length of the same keratin fiber, which is generally differently sensitized (i.e. damaged) between its end and its root.

The dyeing is generally performed in a strongly alkaline medium, in the presence of hydrogen peroxide. However, the use of alkaline media in the presence of hydrogen peroxide may have the drawback of causing considerable degradation of the fibers, and bleaching of keratin fibers, which is not always desirable.

Furthermore, this type of composition may have the drawback of involving the mixing of the aqueous hydrogen peroxide solution and the dye support at the time of application of the composition to the keratin fibers.

The oxidation dyeing of keratin fibers may also be performed using oxidizing systems other than hydrogen peroxide, such as enzymatic systems, for example, with enzymes of the 2 electron oxidase type. French patent application FR 2 769 219 describes the use of a uricase enzyme and its uric acid substrate in oxidation dyeing to dye keratin fibers. These enzymes catalyze the oxidation of a substrate by atmospheric oxygenation to generate one or more oxidation products and the aqueous hydrogen peroxide solution. The aqueous hydrogen peroxide solution generated may be used to oxidize oxidation dye precursors and, consequently, produce the color on the hair. This system makes it possible to conduct oxidation dyeing without mixing at the time of use. However, the dye formulations using alcohol oxidase, although used under conditions that do not result in hair degradation compatible to that caused by formulations using aqueous hydrogen peroxide solution, and although offering the possibility of being formulated all-in-one, may lead to colorations that are insufficient with respect to the homogeneity of the color, the dyeing power and the chromaticity.

To overcome at least one of the above problems, disclosed herein are novel compositions for dyeing keratin fibers by oxidation dyeing, which respect the nature of the keratin fiber, can offer the possibility of being formulated all-in-one, and can lead to strong, homogeneous colors and high chromaticity.

The present inventor has discovered novel compositions comprising at least, one enzyme of alcohol oxidase type as an oxidizing system, at least one substrate for the enzyme and at least one cationic oxidation base. The compositions disclosed herein can produce strong, chromatic, unselective and fast colors, and are capable of giving varied shades of intense and uniform color, without any significant degradation of the hair.

Other characteristics, aspects, subjects and advantages of the present invention will emerge even more clearly on reading the description and the examples that follow.

The term “cationic oxidation base” means an oxidation base bearing at least one permanent cationic charge. The cationic oxidation bases that may be used herein may be chosen from para-phenylenediamines as described in patent applications FR 2 766 177 and FR 2 766 178, para-aminophenols as described in patent applications FR 2 766 177 and FR 2 766 178, ortho-phenylenediamines as described in patent applications FR 2 782 718, FR 2 782 716 and FR 2 782 719, and cationic ortho-aminophenols, heterocyclic bases and double bases as described in patent application FR 2 766 179. For example, the cationic heterocyclic bases are chosen from cationic pyrazole bases and cationic pyrazolopyrimidines (as described in, for example, French patents FR 2 788 521 and FR 2 788 522). The cationic non-heterocyclic bases are chosen, for example, from para-phenylenediamines. In one embodiment, the cationic oxidation bases as disclosed herein are chosen from para-phenylenediamines comprising at least one quaternized nitrogen atom, wherein one of the amine functional groups is a tertiary amine bearing a pyrrolidine nucleus.

Among the cationic oxidation bases, mention may be made, for example, of the compounds of formula (I): 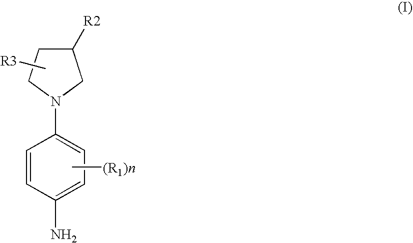
wherein:
n ranges from 0 to 4, wherein when n is greater than or equal to 2, then the radicals R₁ may be identical or different,

R₁ is chosen from halogen atoms, an onium radical Z, and C₁-C₆ aliphatic and acyclic, saturated and unsaturated, hydrocarbon-based chains, which may comprise at least one entity chosen from oxygen, nitrogen, silicon and sulfur atoms and an SO₂ group, and which may be substituted with at least one radical chosen from hydroxyl and amino radicals, provided that the radical R₁ does not comprise a peroxide bond or a diazo, nitro or nitroso radical,
R₂ is chosen from an onium radical Z and a radical —X—C═N⁺(R₈R₈′)—NR₉R₁₀ wherein X is chosen from an oxygen atom and a radical —NR₁₁, and R₈, R₈′, R₉, R₁₀ and R₁₁, which may be identical or different, are each chosen from a hydrogen atom, C₁-C₄ alkyl radicals and C₁-C₄ hydroxyalkyl radicals,
R₃ is chosen from a hydrogen atom and a hydroxyl radical.

The term “onium radical” means a quaternary radical of a nitrogenous base.

Among the onium radical Z of the groups R₁ and R₂ mention may be made of formula (II) below: 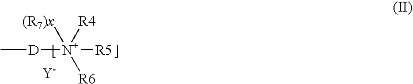
wherein D is chosen from a single bond and linear and branched C₁-C₁₄ alkylene chains which may comprise at least one hetero atom chosen from oxygen, sulfur and nitrogen, and which may be substituted with at least one radical chosen from hydroxyl, C₁-C₆ alkoxy and amino radicals, and which may bear at least one ketone functional group,
R₄, R₅ and R₆, which may be identical or different, are each chosen from C₁-C₁₅ alkyl radicals, C₁-C₆ monohydroxyalkyl radicals, C₂-C₆ polyhydroxyalkyl radicals, (C₁-C₆)alkoxy(C₁-C₆)alkyl radicals, aryl radicals, benzyl radicals, C₁-C₆ amidoalkyl radicals, tri(C₁-C₆)alkylsilane(C₁-C₆)alkyl radicals, C₁-C₆ aminoalkyl radicals, C₁-C₆ aminoalkyl radicals in which the amine is mono- or disubstituted with at least one radical chosen from C₁-C₄ alkyl, (C₁-C₆)alkylcarbonyl, amido and (C₁-C₆)alkylsulfonyl radicals; or R₄, R₅ and R₆ together, in pairs, form, with the nitrogen atom to which they are attached, a 4-, 5-, 6- or 7-membered carbon-based saturated ring optionally comprising at least one hetero atom, for example, an azetidine ring, a pyrrolidine ring, a piperidine ring, a piperazine ring or a morpholine ring, wherein the cationic ring may be substituted with at least one entity chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, (C₁-C₆)alkylcarbonyl radicals, a thio (—SH) radical, C₁-C₆ thioalkyl (—R—SH) radicals, (C₁-C₆)alkylthio radicals, an amino radical, and an amino radical mono- or disubstituted with at least one radical chosen from (C₁-C₆)alkyl, (C₁-C₆)alkylcarbonyl, amido and (C₁-C₆)alkylsulfonyl radicals,
R₇ is chosen from C₁-C₆ alkyl radicals, C₁-C₆ monohydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is mono- or disubstituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)alkylsulfonyl(C₁-C₆)alkyl radicals, (C₁-C₆)alkylcarbonyl(C₁-C₆)alkyl radicals, N-(C₁-C₆)alkylcarbamyl(C₁-C₆)alkyl radicals, and N-(C₁-C₆)alkylsulfonamido(C₁-C₆)alkyl radicals,
x is 0 or 1.

When x=0, then the linker arm D is attached to the nitrogen atom bearing the radicals R₄ to R₆.

When x=1, then two of the radicals R₄ to R₆ form, together with the nitrogen atom to which they are attached, a 4-, 5-, 6- or 7-membered saturated ring and D is linked to a carbon atom of the saturated ring.

Y⁻ is a counterion.

The radicals R₁ and R₂, which may be identical or different, may also be the onium radical Z corresponding to formula (III): 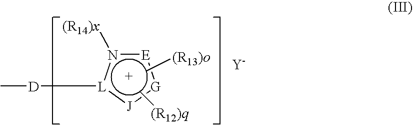
wherein D has the same meaning as defined above,
the ring members E, G, J and L, which may be identical or different, are each chosen from carbon, oxygen, sulfur and nitrogen atoms to form a pyrrole, pyrazole, imidazole, triazole, oxazole, isooxazole, thiazole or isothiazole ring,
q is an integer ranging from 0 to 4,
o is an integer ranging from 0 to 3,
q+o is an integer ranging from 0 to 4,
the radicals R₁₂, which may be identical or different, are each chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, C₁-C₆ alkylcarbonyl radicals, a thio radical, C₁-C₆ thioalkyl radicals, (C₁-C₆)alkylthio radicals, an amino radical, and an amino radical mono- or disubstituted with at least one radical chosen from (C₁-C₆)alkyl, (C₁-C₆)alkylcarbonyl, amido and (C₁-C₆)alkylsulfonyl radicals; C₁-C₆ monohydroxyalkyl radicals and C₂-C₆ polyhydroxyalkyl radicals, wherein the radicals R₁₂ are borne by a carbon atom;
the radicals R₁₃, which may be identical or different, are chosen from C₁-C₆ alkyl radicals, C₁-C₆ monohydroxyalkyl radicals, C₂-C₆ polyhydroxyalkyl radicals, tri(C₁-C₆)alkylsilane(C₁-C₆)alkyl radicals, (C₁-C₆)alkylcarboxy(C₁-C₆)alkyl radicals and benzyl radicals, wherein the radicals R₁₃ are borne by a nitrogen atom;
the radical R₁₄ is chosen from C₁-C₆ alkyl radicals, C₁-C₆ mono-hydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is substituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)-alkylsulfinyl(C₁-C₆)alkyl radicals, (C₁-C₆)alkylsulfonyl(C₁-C₆)alkyl radicals, (C₁-C₆)alkyl-carbonyl(C₁-C₆)alkyl radicals, N-(C₁-C₆)alkylcarbamyl(C₁-C₆)alkyl radicals, and N-(C₁-C₆)alkylsulfonamido(C₁-C₆)alkyl radicals,
x=0 or 1.

When x=0, the linker arm D is attached to the nitrogen atom.

When x=1, the linker arm D is attached to one of the ring members E, G, J or L.

Y⁻ is a counterion.

The radicals R₁ and R₂, which may be identical or different, may also be the onium radical Z corresponding to formula (IV): 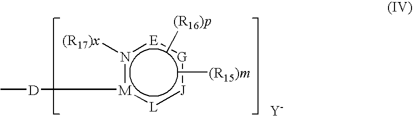
wherein D has the same meaning as defined above,
the ring members E, G, J, L and M, which may be identical or different, are each chosen from carbon, oxygen, sulfur and nitrogen atoms so as to form a pyridine, pyrimidine, pyrazine, triazine or pyridazine ring,
p is an integer ranging from 0 to 3,
m is an integer ranging from 0 to 5,
p+m is an integer ranging from 0 to 5,
the radicals R₁₅, which may be identical or different, are each chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, C₁-C₆ alkylcarbonyl radicals, a thio radical, C₁-C₆ thioalkyl radicals, (C₁-C₆)alkylthio radicals, an amino radical, an amino radical substituted with at least one radical chosen from (C₁-C₆)alkyl, (C₁-C₆)alkylcarbonyl, amido and (C₁-C₆)alkylsulfonyl radicals; C₁-C₆ monohydroxyalkyl radicals and C₂-C₆ polyhydroxyalkyl radicals, wherein the radicals R₁₅ are borne by a carbon atom;
the radicals R₁₆, which may be identical or different, are each chosen from C₁-C₆ alkyl radicals, C₁-C₆ monohydroxyalkyl radicals, C₂-C₆ polyhydroxyalkyl radicals, tri(C₁-C₆)alkylsilane(C₁-C₆)alkyl radicals, (C₁-C₆)alkoxy(C₁-C₆)alkyl radicals, C₁-C₆ carbamylalkyl radicals, (C₁-C₆)alkylcarboxy(C₁-C₆)alkyl radicals and benzyl radicals, wherein the radicals R₁₆ are borne by a nitrogen atom;
the radical R₁₇ is chosen from C₁-C₆ alkyl radicals, C₁-C₆ mono-hydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is mono- or disubstituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)alkylsulfinyl(C₁-C₆)alkyl radicals, (C₁-C₆)alkylsulfonyl(C₁-C₆)alkyl radicals, (C₁-C₆)alkylcarbonyl(C₁-C₆)alkyl radicals, N-(C₁-C₆)alkylcarbamyl(C₁-C₆)alkyl radicals, and N-(C₁-C₆)alkylsulfonamido(C₁-C₆)alkyl radicals,
x is 0 or 1.

When x=0, the linker arm D is attached to the nitrogen atom.

When x=1, the linker arm D is attached to one of the ring members E, G, J, L or M.

Y⁻ is a counterion.

The radicals R₁ and R₂, which may be identical or different, may also be a radical of the onium type of formula:
—X—P(O)(O⁻)OCH₂CH₂N⁺(CH₃)₃
wherein X is chosen from an oxygen atom and a radical —NR₁₈, wherein R₁₈ is chosen from a hydrogen atom, C₁-C₄ alkyl radicals and hydroxyalkyl radicals.

The radicals R₁ and R₂, which may be identical or different, may also be a radical of the formula —X—C═N⁺(R₁₉R′₁₉)—NR₂₀R₂₁ wherein X is chosen from an oxygen atom and a radical —NR₂₂, R₁₉, R′₁₉, R₂₀, R₂₁ and R₂₂, which may be identical or different, are each chosen from a hydrogen atom, C₁-C₄ alkyl radicals and hydroxyalkyl radicals.

As disclosed herein, the cationic para-phenylenediamines having the following exemplary formulae are used:

| Formula | Nomenclature |
|---|---|
| 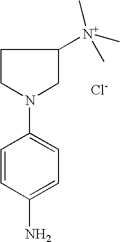 | [1-(4-Amino-phenyl)-pyr- rolidin-3-yl]-trimethyl-ammonium chloride (1) |
| 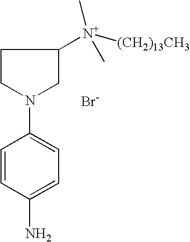 | [1-(4-Amino-phenyl)-pyr- rolidin-3-yl]-di- methyl-tetradecyl-ammonium bromide (2) |
| 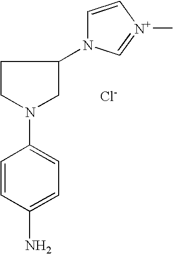 | 3-[1-(4-Amino-phenyl)-pyr- rolidin-3-yl]-1-methyl-3H-imi- dazol-1-ium chloride (5) |
| 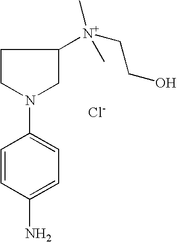 | [1-(4-Amino-phenyl)-pyr- rolidin-3-yl]-(2-hy- droxy-ethyl)-di- methyl-ammonium chloride (6) |
| 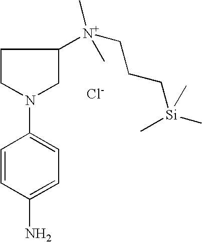 | [1-(4-Amino-phenyl)-pyr- rolidin-3-yl]-dimethyl-(3-tri- methylsilanyl-propyl)-ammonium chloride (7) |
| 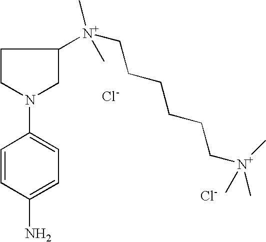 | [1-(4-Amino-phenyl)-pyr- rolidin-3-yl] [symbol] tri- methylammonium-hexyl)-di- methyl-ammonium dichloride (8) |
| 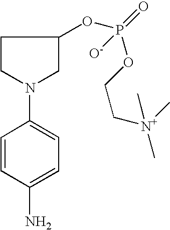 | [1-(4-Amino-phenyl)-pyr- rolidin-3-yl]-oxo- phosphorylcholine (9) |
| 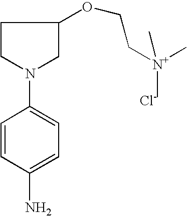 | {2-[1-(4-Amino-phe- nyl)-pyrrolidin-3-yl- oxy]-ethyl}-tri- methyl-ammonium chloride (10) |
| 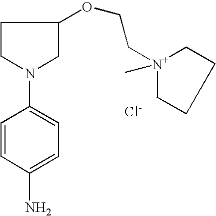 | 1-{2-[1-(4-Amino-phenyl)-pyr- rolidin-3-yloxy]-ethyl}-1-meth- yl-pyrrolidinium chloride (11) |
| 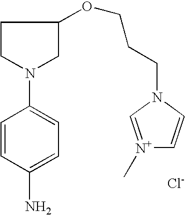 | 3-{3-[1-(4-Amino-phe- nyl)-pyrrolidin-3-yl- oxy]-propyl}-1-meth- yl-3H-imidazol-1-ium chloride (12) |
| 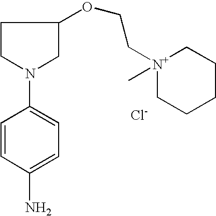 | 1-{2-[1-(4-Amino-phenyl)-pyr- rolidin-3-yloxy]-ethyl}-1-meth- yl-piperidinium chloride (13) |
| 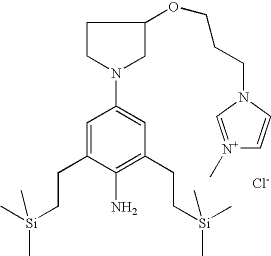 | 3-{3-[1-(5-tri- methylsilanylethyl-4-Amino-3-tri- methylsilanylethyl-phe- nyl)-pyrrolidin-3-yl- oxy]-propyl}-1-meth- yl-3H-imidazol-1-um chloride (14) |
| 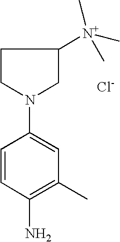 | [1-(4-Amino-3-methyl-phe- nyl)-pyrrolidin-3-yl]-tri- methyl-ammonium chloride (15) |
| 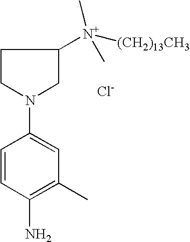 | [1-(4-Amino-3-methyl-phe- nyl)-pyrrolidin-3-yl]-di- methyl-tetradecyl-ammonium chloride (16) |
| 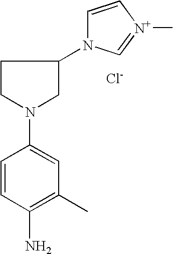 | 3-[1-(4-Amino-3-methyl-phe- nyl)-pyrrolidin-3-yl]-1-meth- yl-3H-imidazol-1-ium chloride (19) |
| 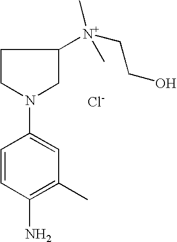 | [1-(4-Amino-3-methyl-phe- nyl)-pyrrolidin-3-yl]-(2-hy- droxy-ethyl)-di- methyl-ammonium chloride (20) |
| 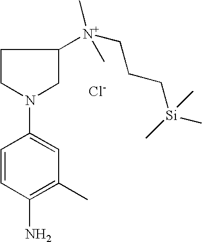 | [1-(4-Amino-3-methyl-phe- nyl)-pyrrolidin-3-yl]-di- methyl-(3-tri- methylsilanyl-propyl ammonium chloride (21) |
| 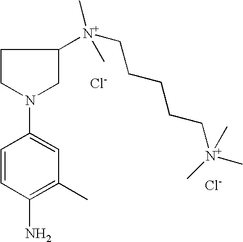 | [1-(4-Amino-3-methyl-phe- nyl)-pyrrolidin-3-yl] [symbol] tri- methylammonium-hex- yl)-dimethyl-ammonium dichloride (22) |
| 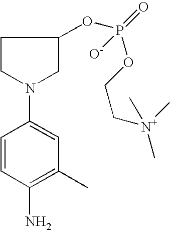 | [1-(4-Amino-3-methyl-phe- nyl)-pyrrolidin-3-yl]-oxo- phosphorylcholine (23) |
| 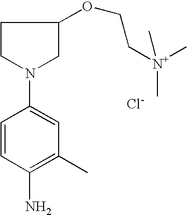 | {2-[1-(4-Amino-3-meth- yl-phenyl)-pyr- rolidin-3-yloxy]-eth- yl}-trimethyl-ammonim chloride (24) |
| 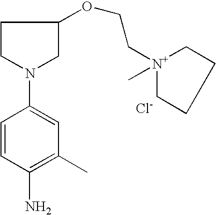 | 1-{2-[1-(4-Amino-3-meth- yl-phenyl)-pyrrolidin-3-yl- oxy]-ethyl}-1-methyl-pyrrolidinium chloride (25) |
| 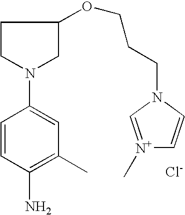 | 3-{3-[1-(4-Amino-3-meth- yl-phenyl)-pyr- rolidin-3-yloxy]-pro- pyl}-1-methyl-3H-imi- dazol-1-um chloride (26) |
| 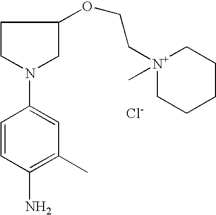 | 1-{2-[1-(4-Amino-3-meth- yl-phenyl)-pyrrolidin-3-yl- oxy]-ethyl}-1-methyl-pipe- ridinium chloride (27) |
| 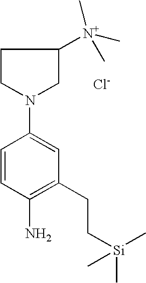 | [1-(4-Amino-3-tri- methylsilanylethyl-phe- nyl)-pyrrolidin-3-yl]-tri- methyl-ammonium chloride (28) |
| 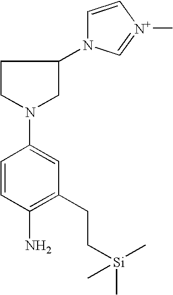 | 3-[1-(4-Amino-3-tri- methylsilanylethyl-phe- nyl)-pyrrolidin-3-yl]-1-meth- yl-3H-imidazol-1-ium chloride (29) |
| 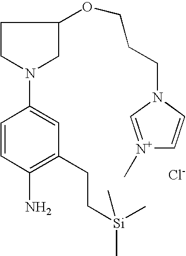 | 3-{3-[1-(4-Amino-3-tri- methylsilanylethyl-phe- nyl)-pyrrolidin-3-yl- oxy]-propyl}-1-meth- yl-3H-imidazol-1-um chloride (30) |
| 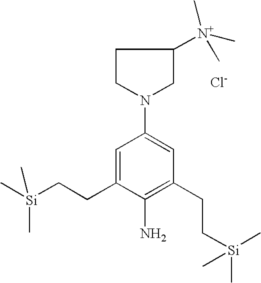 | [1-(5-trimethylsilanylethyl-4-Amino-3-tri- methylsilanylethyl-phe- nyl)-pyrrolidin-3-yl]-tri- methyl-ammonium chloride (31) |
| 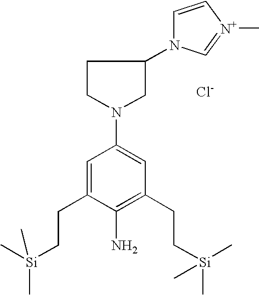 | 3-[1-(5-tri- methylsilanylethyl-4-Amino-3-tri- methylsilanylethyl-phe- nyl)-pyrrolidin-3-yl]-1-meth- yl-3H-imidazol-1-ium chloride (32) |
| 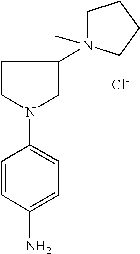 | 1′-(4-Amino-phenyl)-1-meth- yl-[1,3′]bipyrrolidinyl-1-ium chloride (33) |
| 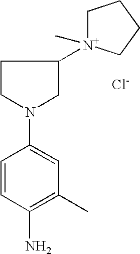 | 1′-(4-Amino-3-methyl-phe- nyl)-1-methyl-[1,3′]bi- pyrrolidinyl-1-ium chloride (34) |
| 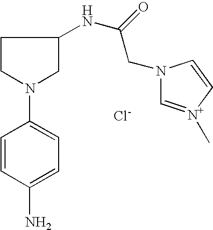 | 3-{[1-(4-amino-phenyl)-pyr- rolidin-3-ylcarbamoyl]-meth- yl}-1-methyl-3H-imi- dazol-1-ium chloride (35) |
| 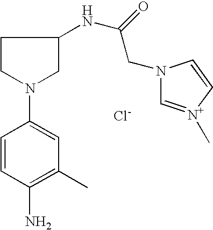 | 3-{[1-(4-amino-3-meth- yl-phenyl)-pyr- rolidin-3-yl- carbamoyl]-methyl}-1-meth- yl-3H-imidazol-1-ium chloride (36) |
| 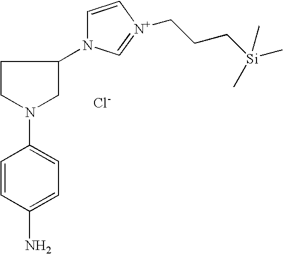 | 3-[1-(4-amino-phenyl)-pyr- rolidin-3-yl]-1-(3-tri- methylsilanyl-propyl)-3H-imi- dazol-1-ium chloride (37) |
| 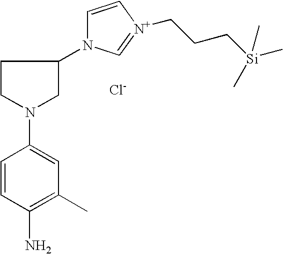 | 3-[1-(4-amino-phenyl)-pyr- rolidin-3-yl]-1-(3-tri- methylsilanyl-pro- pyl)-3H-imidazol-1-ium chloride (38) |
| 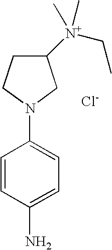 | [1-(4-amino-phenyl)-pyr- rolidin-3-yl]-ethyl- dimethyl-ammonium chloride (39) |
| 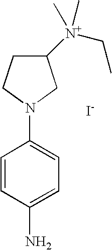 | [1-(4-amino-phenyl)-pyr- rolidin-3-yl]-ethyl- dimethyl-ammonium iodide (40) |
| 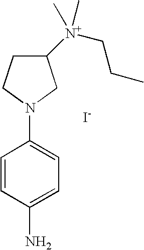 | [1-(4-amino-phenyl)-pyr- rolidin-3-yl]-propyl- dimethyl-ammonium iodide, (41) |
| 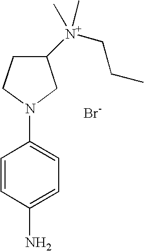 | [1-(4-amino-phenyl)-pyr- rolidin-3-yl]-propyl- dimethyl-ammonium bromide (42) |
| 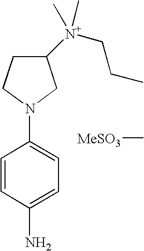 | [1-(4-amino-phenyl)-pyr- rolidin-3-yl]-propyl- dimethyl-ammonium methosulfate (43) |
| 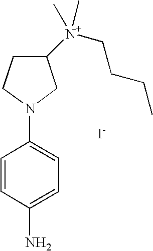 | [1-(4-amino-phenyl)-pyr- rolidin-3-yl]-butyl- dimethyl-ammonium iodide (44) |
| 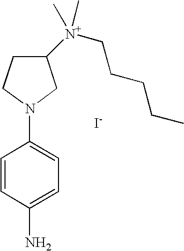 | [1-(4-amino-phenyl)-pyr- rolidin-3-yl]-pentyl- dimethyl-ammonium iodide (45) |
| 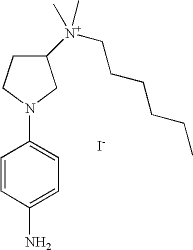 | [1-(4-amino-phenyl)-pyr- rolidin-3-yl]-hexyl- dimethyl-ammonium iodide (46) |
| 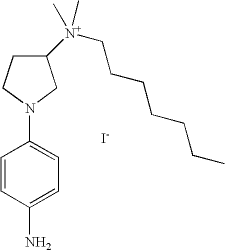 | [1-(4-amino-phenyl)-pyr- rolidin-3-yl]-heptyl- dimethyl-ammonium iodide (47) |
| 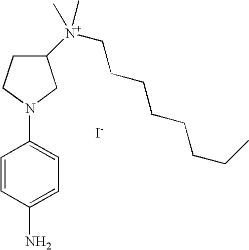 | [1-(4-amino-phenyl)-pyr- rolidin-3-yl]-octyl- dimethyl-ammonium iodide (48) |
| 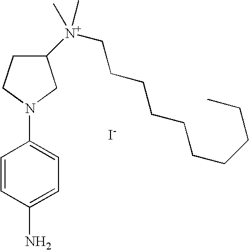 | [1-(4-amino-phenyl)-pyr- rolidin-3-yl]-decyl- dimethyl-ammonium iodide (49) |
| 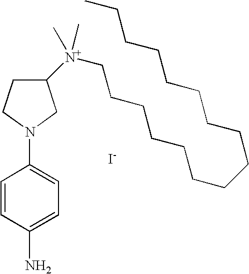 | [1-(4-amino-phenyl)-pyr- rolidin-3-yl]-hexa- decyldimethyl-ammonium iodide (50) |
| 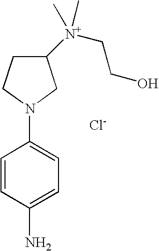 | [1-(4-amino-phenyl)-pyr- rolidin-3-yl]-hydroxy- ethyldimethyl-ammonium chloride (51) |
| 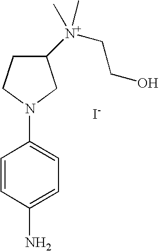 | [1-(4-amino-phenyl)-pyr- rolidin-3-yl]-hy- droxyethyldimethyl-ammonium iodide (52) |

The at least one cationic oxidation base is present in an amount ranging, for example, from 0.001% to 10% by weight, such as from 0.005% to 6% by weight, relative to the total weight of the composition.

As disclosed herein, the alcohol oxidase enzymes that are used in the dye composition in accordance with the present disclosure belong to the category EC 1.1.3 of the enzyme nomenclature (see Enzyme Nomenclature, Academic Press Inc, 1992).

The alcohol oxidase enzymes are chosen, for example, from primary alcohol oxidases (EC 1.1.3.13), secondary alcohol oxidases (EC 1.1.3.18), long hydrocarbon chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7), also known as aryl alcohol oxidases.

In one embodiment, the enzyme used in the composition as disclosed herein is a primary alcohol oxidase (EC1.1.3.13).

The alcohol oxidase enzymes form, for example, a category of 2-electron oxidoreductase enzymes.

The alcohol oxidase enzyme used in the dye composition as disclosed herein may be derived from a plant, animal or microorganism (bacterium, fungus, yeast, microalga or virus) extract, from differentiated or undifferentiated cells, obtained in vivo or in vitro, which are genetically modified or unmodified, or synthetic (obtained via chemical or biotechnological synthesis).

Examples that may be mentioned include, for example, enzymes extracted from the following species: Pinus, Gastropode, Manduca, Pichia, Candida, Pleurotus, Pseudomonas, Rhodococcus, Aspergillus, Kamagataella, Phanerochaete, Polyporus, Hansenula, Poria, Penicillium. For example, the enzymes used herein are extracted from the following species: Pinus strobus, which is a species of plant origin, Gastropode mollusc, Manduca sexta, which are of animal origin, Pichia sp. (pastoris, methanolica, angusta) and Candida sp. (boidinii, albicans, tropicalis), which are yeasts, Pleurotus pulmonarius Aspergillus niger, Kamagataella pastoris, Phanerochaete chrysosporium, Polyporus obtusus, Hansenula polymorpha, Poria contigua, Penicillium simplicissimum, which are fungi, and Pseudomonas pseudoalcaligenes, Rhodococcus erythropolis, which are bacteria.

In one embodiment, the alcohol oxidase used in the composition as disclosed herein is derived from the strain Pichia pastoris.

Generally, the at least one alcohol oxidase enzyme used in the dye composition is present in an amount ranging, for example, from 0.05% and 20% by weight, such as from 0.1% to 10% by weight, and further such as from 0.5% to 8% by weight, relative to the total weight of the dye composition.

The enzymatic activity of the alcohol oxidase enzymes used herein may be defined from the oxidation of the donor under aerobic conditions. The unit U corresponds to the amount of enzyme leading to the generation of 1 μmol of hydrogen peroxide per minute at given pH and at a temperature of 25° C.

For example, the amount of alcohol oxidase enzyme used in the dye composition ranges from 10³ U to 10⁵ U, such as from 2×10³ U to 2×10⁴ Upper 100 g of dye composition.

The at least one substrate for the enzyme is also known as donors for the enzyme. The nature of this substrate varies as a function of the nature of the alcohol oxidase enzyme used. The substrate for the enzyme in the compositions as disclosed herein is, for example, an alcohol chosen from branched and unbranched, saturated and unsaturated, substituted and unsubstituted, primary and secondary alcohols, long hydrocarbon chain alcohols and aromatic alcohols. For example, mention may be made, as donors for the primary alcohol oxidases, of primary alcohols comprising from 1 to 6 carbon atoms; as donors for the aryl alcohol oxidases: benzyl alcohol, 4-tert-butylbenzyl alcohol, 3-hydroxy-4-methoxybenzyl alcohol, veratryl alcohol, 4-methoxy-benzyl alcohol, cinnamyl alcohol; 2,4-hexadien-1-ol may also be used as donors for the aryl alcohol oxidases.

According to another variant, the substrate for the enzyme is a compound bearing at least one alcohol functional group chosen from aliphatic and aromatic alcohol functional groups, suitable for reaction with the enzyme used. The compound bearing at least one alcohol functional group chosen from aliphatic and aromatic alcohol functional groups may, for example, be an oxidation dye precursor or a cosmetically acceptable adjuvant, for example, a polymer, a surfactant or a preserving agent bearing at least one alcohol functional group. In one embodiment, the substrate for the enzyme is an oxidation dye precursor bearing at least one alcohol functional group chosen from aliphatic and aromatic alcohol functional groups. For example, N—(β-hydroxypropyl)-para-phenylenediamine, which bears a primary alcohol functional group, may serve as oxidation base and as substrate for the alcohol oxidase. Similarly, couplers, such as meta- or para-aminophenol, may serve the two functions. Such couplers are described hereinbelow. In this variant, the use of other substrates for the enzyme is optional.

Thus, disclosed herein is a composition for dyeing keratin fibers, for example, human keratin fibers such as the hair, comprising, in a medium suitable for dyeing, at least one cationic oxidation base; at least one alcohol oxidase enzyme; at least one substrate bearing at least one alcohol functional group for the enzyme, wherein the at least one substrate may be replaced totally or partially by the at least one cationic oxidation base in the case where the at least one cationic oxidation base bears at least one alcohol functional group chosen from aliphatic and aromatic alcohol functional groups.

By using the composition as disclosed herein, the risks associated with the handling of hydrogen peroxide can be reduced. Furthermore, the concentration of preserving agents in the compositions disclosed herein may be reduced by providing compounds comprising at least one alcohol functional group that also have preserving properties.

Generally, the at least one substrate for the enzyme is present in an amount ranging, for example, from 0.01% to 60% by weight, such as from 0.05% to 30% by weight, relative to the total weight of the composition.

The composition as disclosed herein may also comprise at least one non-cationic oxidation base, which may be chosen, for example, from para-phenylenediamines, bis(phenyl)alkylenediamines, para-aminophenols, ortho-aminophenols and heterocyclic bases, and the addition salts thereof.

Among the para-phenylenediamines, mention may be made, for example, of para-phenylenediamine, para-tolylenediamine, 2-chloro-para-phenylenediamine, 2,3-dimethyl-para-phenylenediamine, 2,6-dimethyl-para-phenylenediamine, 2,6-diethyl-para-phenylenediamine, 2,5-dimethyl-para-phenylenediamine, N,N-dimethyl-para-phenylenediamine, N,N-diethyl-para-phenylenediamine, N,N-dipropyl-para-phenylenediamine, 4-amino-N,N-diethyl-3-methylaniline, N,N-bis(β-hydroxyethyl)-para-phenylenediamine, 4-amino-N,N-bis(β-hydroxyethyl)-2-methylaniline, 4-amino-2-chloro-N,N-bis(β-hydroxyethyl)aniline, 2-β-hydroxyethyl-para-phenylenediamine, 2-fluoro-para-phenylenediamine, 2-isopropyl-para-phenylenediamine, N-(β-hydroxypropyl)-para-phenylenediamine, 2-hydroxymethyl-para-phenylenediamine, N,N-dimethyl-3-methyl-para-phenylenediamine, N-ethyl-,N-(β-hydroxyethyl)-para-phenylenediamine, N-(β,γ-dihydroxypropyl)-para-phenylenediamine, N-(4′-aminophenyl)-para-phenylenediamine, N-phenyl-para-phenylenediamine, 2-β-hydroxyethyloxy-para-phenylenediamine, 2-β-acetylaminoethyloxy-para-phenylenediamine, N-(β-methoxyethyl)-para-phenylene-diamine, 4-aminophenylpyrrolidine, 2-thienyl-para-phenylenediamine and 2-β-hydroxyethylamino-5-aminotoluene, and the acid addition salts thereof.

Among the para-phenylenediamines mentioned above, para-phenylenediamine, para-tolylenediamine, 2-isopropyl-para-phenylenediamine, 2-β-hydroxyethyl-para-phenylenediamine, 2-β-hydroxyethyloxy-para-phenylenediamine, 2,6-dimethyl-para-phenylenediamine, 2,6-diethyl-para-phenylenediamine, 2,3-dimethyl-para-phenylenediamine, N,N-bis(β-hydroxyethyl)-para-phenylenediamine, 2-chloro-para-phenylenediamine and 2-β-acetylaminoethyloxy-para-phenylenediamine and the acid addition salts thereof may, for example, be used.

Among the bis(phenyl)alkylenediamines, mention may be made, for example, of N,N′-bis(β-hydroxyethyl)-N,N′-bis(4′-aminophenyl)-1,3-diaminopropanol, N,N′-bis(β-hydroxyethyl)-N,N′-bis(4′-aminophenyl)ethylenediamine, N,N′-bis(4-aminophenyl)tetramethylenediamine, N,N′-bis(β-hydroxyethyl)-N,N′-bis(4-aminophenyl)tetramethylenediamine, N,N′-bis(4-methylaminophenyl)tetramethylene-diamine, N,N′-bis(ethyl)-N,N′-bis(4′-amino-3′-methylphenyl)ethylenediamine and 1,8-bis(2,5-diaminophenoxy)-3,6-dioxaoctane, and the acid addition salts thereof.

Among the para-aminophenols, mention may be made, for example, of para-aminophenol, 4-amino-3-methylphenol, 4-amino-3-fluorophenol, 4-amino-3-hydroxymethylphenol, 4-amino-2-methylphenol, 4-amino-2-hydroxymethylphenol, 4-amino-2-methoxymethylphenol, 4-amino-2-aminomethylphenol, 4-amino-2-(β-hydroxyethylaminomethyl)phenol and 4-amino-2-fluorophenol, 4-amino-2,6-dichlorophenol, 4-amino-6[(5′-amino-2′-hydroxy-3′-methyl)phenyl-methyl]2-methylphenol, bis(5′-amino-2′-hydroxyl)-phenylmethane and the acid addition salts thereof.

Among the ortho-aminophenols, mention may be made, for example, of 2-aminophenol, 2-amino-5-methylphenol, 2-amino-6-methylphenol and 5-acetamido-2-aminophenol, and the acid addition salts thereof.

Among the heterocyclic bases, mention may be made, for example, of pyridine derivatives, pyrimidine derivatives and pyrazole derivatives.

Among the pyridine derivatives, mention may be made of the compounds described, for example, in British patents GB 1 026 978 and GB 1 153 196, such as 2,5-diaminopyridine, 2-(4-methoxyphenyl)amino-3-aminopyridine, and 3,4-diaminopyridine, and the acid addition salts thereof. Other pyridine oxidation bases that may be used herein include, for example, the 3-aminopyrazolo[1,5-a]pyridine oxidation bases and the addition salts thereof described, for example, in patent application FR 2 801 308. Examples that may be mentioned include pyrazolo[1,5-a]pyrid-3-ylamine; 2-acetylaminopyrazolo-[1,5-a]pyrid-3-ylamine; 2-morpholin-4-ylpyrazolo[1,5-a]pyrid-3-ylamine; 3-aminopyrazolo[1,5-a]pyrid-2-carboxylic acid; 2-methoxypyrazolo[1,5-a]pyrid-3-ylamino; (3-aminopyrazolo[1,5-a]pyrid-7-yl)methanol; 2-(3-aminopyrazolo[1,5-a]pyrid-5-yl)ethanol; 2-(3-aminopyrazolo[1,5-a]pyrid-7-yl)ethanol; (3-aminopyrazolo[1,5-a]pyrid-2-yl)methanol; 3,6-diaminopyrazolo[1,5-a]pyridine; 3,4-diaminopyrazolo[1,5-a]pyridine; pyrazolo[1,5-a]pyridine-3,7-diamine; 7-morpholin-4-ylpyrazolo[1,5-a]pyrid-3-ylamine; pyrazolo[1,5-a]pyridine-3,5-diamine; 5-morpholin-4-ylpyrazolo[1,5-a]pyrid-3-ylamine; 2-[(3-aminopyrazolo[1,5-a]pyrid-5-yl)(2-hydroxyethyl)amino]ethanol; 2-[(3-amino-pyrazolo[1,5-a]pyrid-7-yl)(2-hydroxyethyl)amino]ethanol; 3-aminopyrazolo[1,5-a]pyrid-5-ol; 3-aminopyrazolo[1,5-a]pyrid-4-ol; 3-aminopyrazolo[1,5-a]pyrid-6-ol; 3-aminopyrazolo[1,5-a]pyrid-7-ol; and the acid addition salts thereof.

Among the pyrimidine derivatives, mention may be made, for example, of the compounds described, for example, in patents DE 2 359 399; JP 88-169 571; JP 05 163 124; and EP 0 770 375 or patent application WO 96/15765, such as 2-hydroxy-4,5,6-triaminopyrimidine, 2,4-dihydroxy-5,6-diaminopyrimidine and pyrazolopyrimidine derivatives such as those mentioned in French patent application FR-A-2 750 048, and among which mention may be made, for example, of pyrazolo[1,5-a]pyrimidine-3,7-diamine; 2,5-dimethylpyrazolo[1,5-a]pyrimidine-3,7-diamine; pyrazolo-[1,5-a]pyrimidine-3,5-diamine; 2,7-dimethylpyrazolo[1,5-a]pyrimidine-3,5-diamine; 3-aminopyrazolo[1,5-a]pyrimidin-7-ol; 3-aminopyrazolo[1,5-a]pyrimidin-5-ol; 2-(3-aminopyrazolo[1,5-a]pyrimidin-7-ylamino)ethanol, 2-(7-aminopyrazolo[1,5-a]pyrimidin-3-ylamino)ethanol, 2-[(3-aminopyrazolo[1,5-a]pyrimidin-7-yl)(2-hydroxyethyl)-amino]ethanol, 2-[(7-aminopyrazolo[1,5-a]pyrimidin-3-yl)(2-hydroxyethyl)amino]ethanol, 5,6-dimethylpyrazolo[1,5-a]pyrimidine-3,7-diamine, 2,6-dimethylpyrazolo[1,5-a]pyrimidine-3,7-diamine, 2,5,N7,N7-tetramethylpyrazolo[1,5-a]pyrimidine-3,7-diamine and 3-amino-5-methyl-7-imidazolylpropylaminopyrazolo[1,5-a]pyrimidine, and the acid addition salts thereof, and the tautomeric forms thereof, when a tautomeric equilibrium exists.

Among the pyrazole derivatives, mention may be made, for example, of the compounds described in German patents DE 3 843 892, DE 4 133 957 and patent applications WO 94/08969, WO 94/08970, FR-A-2 733 749 and DE 195 43 988, such as 4,5-diamino-1-methylpyrazole, 4,5-diamino-1-(β-hydroxyethyl)pyrazole, 3,4-diaminopyrazole, 4,5-diamino-1-(4′-chlorobenzyl)pyrazole, 4,5-diamino-1,3-dimethyl-pyrazole, 4,5-diamino-3-methyl-1-phenylpyrazole, 4,5-diamino-1-methyl-3-phenyl-pyrazole, 4-amino-1,3-dimethyl-5-hydrazinopyrazole, 1-benzyl-4,5-diamino-3-methyl-pyrazole, 4,5-diamino-3-tert-butyl-1-methylpyrazole, 4,5-diamino-1-tert-butyl-3-methylpyrazole, 4,5-diamino-1-(β-hydroxyethyl)-3-methylpyrazole, 4,5-diamino-1-ethyl-3-methylpyrazole, 4,5-diamino-1-ethyl-3-(4′-methoxyphenyl)pyrazole, 4,5-diamino-1-ethyl-3-hydroxymethylpyrazole, 4,5-diamino-3-hydroxymethyl-1-methylpyrazole, 4,5-diamino-3-hydroxymethyl-1-isopropyl pyrazole, 4,5-diamino-3-methyl-1-isopropylpyrazole, 4-amino-5-(2′-aminoethyl)amino-1,3-dimethylpyrazole, 3,4,5-triaminopyrazole, 1-methyl-3,4,5-triaminopyrazole, 3,5-diamino-1-methyl-4-methylaminopyrazole and 3,5-diamino-4-(β-hydroxyethyl)amino-1-methylpyrazole, and the acid addition salts thereof.

Generally, the at least one non-cationic oxidation base is present in an amount ranging, for example, from 0.0001% to 20% by weight, such as from 0.005% to 6% by weight, relative to the total weight of the composition.

The dye composition as disclosed herein may also comprise at least one coupler. Among the couplers that may be used, examples include meta-phenylenediamines, meta-aminophenols, meta-diphenols, naphthalene-based couplers and heterocyclic couplers, and the acid and base addition salts thereof.

Examples that may be mentioned include 2-methyl-5-aminophenol, 5-N—(β-hydroxyethyl)amino-2-methylphenol, 6-chloro-2-methyl-5-aminophenol, 3-aminophenol, 1,3-dihydroxybenzene (or resorcinol), 1,3-dihydroxy-2-methylbenzene, 4-chloro-1,3-dihydroxybenzene, 2,4-diamino-1-(β-hydroxyethyloxy)benzene, 2-amino-4-(β-hydroxyethylamino)-1-methoxybenzene, 1,3-diaminobenzene, 1,3-bis(2,4-diaminophenoxy)propane, 3-ureidoaniline, 3-ureido-1-dimethylaminobenzene, sesamol, 1-β-hydroxyethylamino-3,4-methylenedioxybenzene, α-naphthol, 2-methyl-1-naphthol, 6-hydroxyindole, 4-hydroxyindole, 4-hydroxy-N-methylindole, 2-amino-3-hydroxypyridine, 6-hydroxybenzomorpholine, 3,5-diamino-2,6-dimethoxypyridine, 1-N—(β-hydroxyethyl)amino-3,4-methylenedioxybenzene and 2,6-bis(β-hydroxy-ethylamino)toluene, and the acid and base addition salts thereof.

Generally, the at least one coupler is present in an amount ranging, for example, from 0.0001% to 20% by weight, such as from 0.005% to 6% by weight, relative to the total weight of the composition.

In general, the acid addition salts that may be used for the oxidation bases and couplers are chosen, for example, from hydrochlorides, hydrobromides, sulfates, citrates, succinates, tartrates, lactates, tosylates, benzenesulfonates, phosphates and acetates.

The base addition salts that may be used herein are chosen, for example, from the addition salts with sodium hydroxide, potassium hydroxide, ammonia, amines and alkanolamines.

The dye composition as disclosed herein may also comprise at least one direct dye, which may be chosen, for example, from neutral, acidic and cationic nitrobenzene dyes, neutral, acidic and cationic azo direct dyes, neutral, acidic and cationic quinone such as anthraquinone direct dyes, azine direct dyes, methine, azomethine, triarylmethane and indoamine direct dyes and natural direct dyes. In one embodiment, the at least one direct dye is chosen from cationic direct dyes and natural direct dyes.

Among the cationic direct dyes that may be used herein, mention may be made, for example, of the cationic azo direct dyes described in patent applications WO 95/15144, WO 95/01772, and EP-714 954.

Among these compounds, mention may be made, for example, of the following dyes:
-1,3-dimethyl-2-[[4-(dimethylamino)phenyl]azo]-1H-imidazolium chloride,
-1,3-dimethyl-2-[(4-aminophenyl)azo]-1H-imidazolium chloride, and
-1-methyl-4-[(methylphenylhydrazono)methyl]pyridinium methyl sulfate.

Among the natural direct dyes that may be used herein, mention may be made, for example, of lawsone, juglone, alizarin, purpurin, carminic acid, kermesic acid, purpurogallin, protocatechaldehyde, indigo, isatin, curcumin, spinulosin and apigenidin. It is also possible to use extracts or decoctions comprising these natural dyes, such as henna-based poultices or extracts.

The at least one direct dye is present in an amount ranging, for example, from 0.001% to 20% by weight, such as from 0.005% to 10% by weight, relative to the total weight of the composition.

The dye composition as disclosed herein may also comprise at least one adjuvant chosen from various adjuvants conventionally used in hair dye compositions, such as antioxidants, penetrating agents, sequestering agents, fragrances, buffers, dispersants, surfactants, conditioning agents, for instance volatile or non-volatile, modified or unmodified silicones, cationic polymers, cations, film-forming agents, thickening polymers, ceramides, preserving agents, opacifiers, vitamins or provitamins.

The at least one adjuvant is present in an amount ranging, for example, from 0.01% to 20% by weight, relative to the total weight of the composition.

A person skilled in the art will take care to select this or these optional additional compound(s) such that the advantageous properties intrinsically associated with the oxidation dye composition as disclosed herein are not, or are not substantially, adversely affected by the envisaged addition(s).

The medium suitable for dyeing, also known as the dye support, generally comprises water or a mixture of water and at least one organic solvent to dissolve the compounds that would not be sufficiently water-soluble. The at least one organic solvent may, where appropriate, be an enzyme substrate such as ethanol and isopropanol. The at least one organic solvent may also be an enzyme nonsubstrate such as polyol ethers, for instance 2-butoxyethanol, propylene glycol, propylene glycol monomethyl ether and diethylene glycol monomethyl ether and monoethyl ether, phenoxyethanol, and mixtures thereof.

The at least one organic solvent may be present in an amount ranging, for example, from 1% to 40% by weight, such as from 5% to 30% by weight, relative to the total weight of the composition.

The pH of the dye composition as disclosed herein ranges, for example, from 6 to 11, such as from 7 to 10. The pH may be adjusted for the desired value using acidifying or basifying agents commonly used in the dyeing of keratin fibers, or alternatively using standard buffer systems.

Among the acidifying agents that may be mentioned, examples include mineral or organic acids, for instance hydrochloric acid, orthophosphoric acid, sulfuric acid, carboxylic acids, such as acetic acid, tartaric acid, citric acid and lactic acid, and sulfonic acids.

Among the basifying agents that may be mentioned, examples include aqueous ammonia, alkyl metal carbonates, alkanolamines such as monoethanolamine, diethanolamine and triethanolamine and also derivatives thereof, sodium hydroxide, potassium hydroxide and the compounds of formula (V) below: 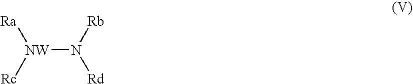
wherein W is a propylene residue optionally substituted with at least one entity chosen from a hydroxyl group and C₁-C₄ alkyl radicals; Ra, Rb, Rc and Rd, which may be identical or different, are each chosen from a hydrogen atom, C₁-C₄ alkyl radicals, and C₁-C₄ hydroxyalkyl radicals.

The dye composition as disclosed herein may be in various forms, such as in the form of liquids, creams or gels, or in any other form that is suitable for dyeing keratin fibers, such as human hair.

When the at least one cationic oxidation dye and the at least one alcohol oxidase are present in the same ready-to-use composition, the composition may, for example, be free of oxygen gas, so as to avoid any premature oxidation of the at least one cationic oxidation dye.

Further disclosed herein is a process for dyeing keratin fibers, for example, human keratin fibers such as the hair, such that when at least one dye composition as disclosed herein is applied to these fibers, the duration of this application is sufficient to develop the desired coloration.

On contact with atmospheric oxygen, the color is revealed by bringing together the alcohol oxidase enzyme and its substrate.

The process comprises applying the composition as disclosed herein to the keratin fibers. After leaving it to act for 3 to 60 minutes, such as from 5 to 40 minutes, the keratin fibers are rinsed, washed with shampoo, rinsed again and then dried.

When the dye composition as disclosed herein is a composition in ready-to-use form, it comprises, in a medium suitable for dyeing keratin fibers, at least one cationic oxidation base, at least one alcohol oxidase enzyme, and at least one substrate for the enzyme. The mixture is then stored in anaerobic form, free of oxygen gas.

According to one variant, this process comprises a preliminary operation comprising separately storing, on the one hand, a composition (A) comprising in a medium suitable for dyeing keratin fibers, at least one cationic oxidation base, and, on the other hand, a composition (B) comprising, in a medium suitable for dyeing keratin fibers, at least one alcohol oxidase enzyme, wherein at least one of the composition (A) and the composition (B) comprises at least one substrate for the enzyme, and then mixing together the compositions (A) and (B) at the time of use before applying this mixture to the keratin fibers.

According to another variant, the process comprises a preliminary operation comprising separately storing, on the one hand, a composition (A) comprising, in a medium suitable for dyeing keratin fibers, at least one substrate for the alcohol oxidase enzyme and at least one cationic oxidation base and, on the other hand, a composition (B) comprising, in a medium suitable for dyeing keratin fibres, at least one alcohol oxidase enzyme, and then mixing together the compositions (A) and (B) at the time of use, before applying this mixture to the keratin fibers.

The color may be developed at acidic, neutral or alkaline pH. In the case where the process is performed using a composition (A) comprising at least one cationic oxidation base and at least one substrate for the enzyme and a composition (B) comprising at least one alcohol oxidase enzyme, the enzyme may be added to the final composition just at the time of use, or may be used starting with the composition (B), which is applied simultaneously with or sequentially to the composition (A).

In this case, the composition (B) (referred to as the oxidizing composition) may also comprise at least one adjuvant chosen from various adjuvants conventionally used in hair dye compositions as defined above.

The pH of the oxidizing composition (B) is such that, after mixing with the dye composition (A), the pH of the resulting composition applied to the keratin fibers ranges, for example, from 6 to 11, such as from 7 to 10. It may be adjusted to the desired value using acidifying or basifying agents, which are commonly used in the dyeing of keratin fibers as defined above.

In one embodiment, the application of the composition as disclosed herein is performed at a temperature ranging from room temperature to 220° C. such as from room temperature to 60° C.

Further disclosed herein is a multi-compartment device or dyeing “kit”, comprising a first compartment comprising the composition (A) as defined above and a second compartment comprising the composition (B) as defined above. This device may be equipped with a means for applying the desired mixture to the hair, such as the devices described in French patent FR 2 586 913.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in this specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contains certain errors necessarily resulting from the standard deviation found in their respective testing measurements.

The example that follows serves to illustrate the invention without, however, being limiting in nature.

### EXAMPLE

The dye composition below was prepared in accordance with the disclosure.

| | |
|---|---|
| [1-(4-amino-phenyl)pyrrolidin-3-yl]trimethyl- | 3 × 10⁻³ mol |
| ammonium chloride | |
| Ethanol (donor substrate) | 25 g |
| meta-Aminophenol (coupler) | 3 × 10⁻³ mol |
| Alcohol oxidase | 20 000 units |
| 2-Amino-2-methyl-1-propanol | qs pH 7 |
| Distilled water | qs 100 g |

The alcohol oxidase used is the product sold by the company Biozyme Laboratories in liquid form at a concentration of 1980 units/ml.

The unit U corresponds to the amount of enzyme leading to the generation of 1 μmol of hydrogen peroxide per minute at pH 7.5 (100 mM phosphate buffer) and at a temperature of 25° C.

The above composition was applied to locks of natural and permanent-waved grey hair containing 90% white hairs, and was left to act for 30 minutes. The bath ratio is set at 5 (the bath ratio is the ratio of the amount of the composition applied over the weight of the locks). The alcohol oxidase was added extemporaneously. The hair was then rinsed, washed with a standard shampoo and then dried.

1. A composition for dyeing keratin fibers, comprising, in a medium suitable for dyeing, at least one cationic oxidation base; at least one alcohol oxidase enzyme; and at least one substrate bearing at least one alcohol functional group for the enzyme, wherein said at least one substrate may be totally or partially replaced by the at least one cationic oxidation base in the case where said at least one cationic oxidation base bears at least one alcohol functional group chosen from aliphatic and aromatic alcohol functional groups.

2. The composition according to claim 1, wherein the keratin fibers are human hair.

3. The composition according to claim 1, wherein the at least one cationic oxidation base is chosen from para-phenylenediamines, para-aminophenols, ortho-aminophenols, ortho-phenylenediamines, double bases and heterocyclic bases and the addition salts thereof.

4. The composition according to claim 3, wherein the heterocyclic bases are chosen from pyrazoles and pyrazolopyrimidines.

5. The composition according to claim 3, wherein the at least one cationic oxidation base is chosen from para-phenylenediamines comprising at least one quaternized nitrogen atom, wherein one of the amine functional groups is a tertiary amine bearing a pyrrolidine nucleus.

6. The composition according to claim 5, wherein the at least one cationic oxidation base is chosen from para-phenylenediamines comprising at least one quaternized nitrogen atom, wherein said para-phenylenediamines comprise an amine group and, in the para position relative to said amine group, a disubstituted amine functional group, the substitutions of which form with the nitrogen atom a pyrrolidine nucleus.

7. The composition according to claim 5, wherein the at least one cationic oxidation base is chosen from compounds of formula (I) below: 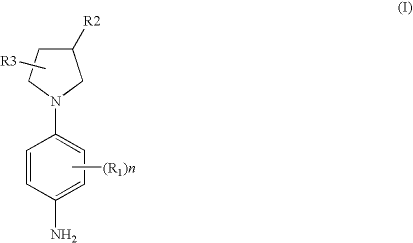
wherein:
n ranges from 0 to 4, wherein when n is greater than or equal to 2, radicals R₁ may be identical or different,
R₁ is chosen from halogen atoms, an onium radical Z, C₁-C₆ aliphatic and alicyclic, saturated and unsaturated hydrocarbon-based chains, which may comprise at least one entity chosen from oxygen, nitrogen, silicon and sulfur atoms and an SO₂ group, and may be substituted with at least one radical chosen from hydroxyl and amino radicals; provided that the radical R₁ does not comprise a peroxide bond or a diazo, nitro or nitroso radical,
R₂ is chosen from an onium radical Z and a radical —X—C═N⁺(R₈R₈′)—NR₉R₁₀ wherein X is chosen from an oxygen atom and a radical —NR₁₁ and R₈, R₈′, R₉, R₁₀ and R₁₁, which may be identical or different, are each chosen from a hydrogen atom, C₁-C₄ alkyl radicals, and C₁-C₄ hydroxyalkyl radicals,
R₃ is chosen from a hydrogen atom and a hydroxyl radical,
the radicals R₁ and R₂, which may be identical or different, may be chosen from the onium radical Z of formula (II) below: 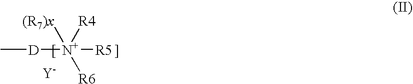
wherein D is chosen from a single bond and linear and branched C₁-C₁₄ alkylene chains, which may comprise at least one hetero atom chosen from oxygen, sulfur and nitrogen, which may be substituted with at least one radical chosen from hydroxyl, C₁-C₆ alkoxy and amino radicals, and which may bear at least one ketone functional group,
R₄, R₅ and R₆, which may be identical or different, are each chosen from C₁-C₁₅ alkyl radicals, C₁-C₆ monohydroxyalkyl radicals, C₂-C₆ polyhydroxyalkyl radicals, (C₁-C₆)alkoxy(C₁-C₆)alkyl radicals, aryl radicals, benzyl radicals, C₁-C₆ amidoalkyl radicals, tri(C₁-C₆)alkylsilane(C₁-C₆)alkyl radicals, C₁-C₆ aminoalkyl radicals, C₁-C₆ aminoalkyl radicals in which the amine is mono- or disubstituted with at least one radical chosen from C₁-C₄ alkyl, (C₁-C₆)alkylcarbonyl, amido and (C₁-C₆)alkylsulfonyl radicals; or R₄, R₅ and R₆ together, in pairs, form, with the nitrogen atom to which they are attached, a 4-, 5-, 6- or 7-membered carbon-based saturated ring optionally comprising at least one hetero atom, wherein the cationic ring may be substituted with at least one entity chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, (C₁-C₆)alkylcarbonyl radicals, a thio radical, C₁-C₆ thioalkyl radicals, (C₁-C₆)alkylthio radicals, an amino radical, and an amino radical mono- or disubstituted with at least one radical chosen from (C₁-C₆)alkyl, (C₁-C₆)alkylcarbonyl, amido and (C₁-C₆)alkylsulfonyl radicals,
R₇ is chosen from C₁-C₆ alkyl radicals, C₁-C₆ monohydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is mono- or disubstituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)-alkylsulfonyl(C₁-C₆)alkyl radicals, (C₁-C₆)alkylcarbonyl(C₁-C₆)alkyl radicals, N-(C₁-C₆)alkylcarbamyl(C₁-C₆)alkyl radicals, and N-(C₁-C₆)alkylsulfonamido(C₁-C₆)alkyl radicals,
x is 0 or 1;
when x=0, then the linker arm D is attached to the nitrogen atom bearing the radicals R₄ to R₆,
when x=1, then two of the radicals R₄ to R₆ form, together with the nitrogen atom to which they are attached, a 4-, 5-, 6- or 7-membered saturated ring and D is linked to a carbon atom of the saturated ring,
Y⁻ is a counterion,
the radicals R₁ and R₂, which may be identical or different, may also be chosen from the onium radical Z of formula (III): 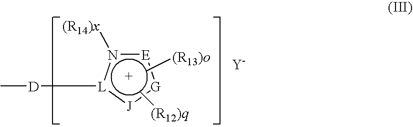
wherein D is chosen from a single bond and linear and branched C₁-C₁₄ alkylene chains, which may comprise at least one hetero atom chosen from oxygen, sulfur and nitrogen, which may be substituted with at least one radical chosen from hydroxyl, C₁-C₆ alkoxy and amino radicals, and which may bear at least one ketone functional group,
ring members E, G, J and L, which may be identical or different, are each chosen from carbon, oxygen, sulfur and nitrogen atoms to form a pyrrole, pyrazole, imidazole, triazole, oxazole, isooxazole, thiazole or isothiazole ring,
q is an integer ranging from 0 to 4,
o is an integer ranging from 0 to 3,
q+o is an integer ranging from 0 to 4,
radicals R₁₂, which may be identical or different, are each chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, C₁-C₆ alkylcarbonyl radicals, a thio radical, C₁-C₆ thioalkyl radicals, (C₁-C₆)alkylthio radicals, an amino radical, an amino radical mono- or disubstituted with at least one radical chosen from (C₁-C₆)alkyl, (C₁C₆)alkylcarbonyl, amido and (C₁-C₆)alkylsulfonyl radicals; C₁-C₆ monohydroxyalkyl radicals and C₂-C₆ polyhydroxyalkyl radicals, wherein the radicals R₁₂ are borne by a carbon atom;
radicals R₁₃, which may be identical or different, are each chosen from C₁-C₆ alkyl radicals, C₁-C₆ monohydroxyalkyl radicals, C₂-C₆ polyhydroxyalkyl radicals, tri(C₁-C₆)alkylsilane(C₁-C₆)alkyl radicals, (C₁-C₆)alkylcarboxy(C₁-C₆)alkyl radicals and benzyl radicals, wherein the radicals R₁₃ are borne by a nitrogen atom;
R₁₄ is chosen from C₁-C₆ alkyl radicals, C₁-C₆ monohydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is substituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)-alkylsulfinyl(C₁-C₆)alkyl radicals, (C₁-C₆)alkylsulfonyl(C₁-C₆)alkyl radicals, (C₁-C₆)alkylcarbonyl(C₁-C₆)alkyl radicals, N-(C₁-C₆)alkylcarbamyl(C₁-C₆)alkyl radicals, and N-(C₁-C₆)alkylsulfonamido(C₁-C₆)alkyl radicals,
x=0 or 1,
when x=0, the linker arm D is attached to the nitrogen atom,
when x=1, the linker arm D is attached to one of the ring members E, G, J or L,
Y⁻ is a counterion,
the radicals R₁ and R₂, which may be identical or different, may also be chosen from the onium radical Z of formula (IV): 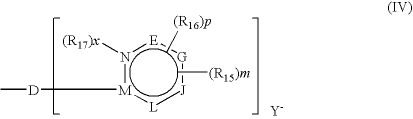
wherein D is chosen from a single bond and linear and branched C₁-C₁₄ alkylene chains, which may comprise at least one hetero atom chosen from oxygen, sulfur and nitrogen, which may be substituted with at least one radical chosen from hydroxyl, C₁-C₆ alkoxy and amino radicals, and which may bear at least one ketone functional group,
ring members E, G, J, L and M, which may be identical or different, are each chosen from carbon, oxygen, sulfur and nitrogen atoms to form a pyridine, pyrimidine, pyrazine, triazine or pyridazine ring,
p is an integer ranging from 0 to 3,
m is an integer ranging from 0 to 5,
p+m is an integer ranging from 0 to 5,
radicals R₁₅, which may be identical or different, are each chosen from halogen atoms, a hydroxyl radical, C₁-C₆ alkyl radicals, C₁-C₆ monohydroxyalkyl radicals, C₂-C₆ polyhydroxyalkyl radicals, C₁-C₆ alkoxy radicals, tri(C₁-C₆)alkylsilane(C₁-C₆)alkyl radicals, an amido radical, a carboxyl radical, C₁-C₆ alkylcarbonyl radicals, a thio radical, C₁-C₆ thioalkyl radicals, (C₁-C₆)alkylthio radicals, an amino radical, an amino radical substituted with at least one radical chosen from (C₁-C₆)alkyl, (C₁-C₆)alkylcarbonyl, amido and (C₁-C₆)alkylsulfonyl radicals; C₁-C₆ monohydroxyalkyl radicals and C₂-C₆ polyhydroxyalkyl radicals, wherein the radicals R₁₅ are borne by a carbon atom,
radicals R₁₆, which may be identical or different, are each chosen from C₁-C₆ alkyl radicals, C₁-C₆ monohydroxyalkyl radicals, C₂-C₆ polyhydroxyalkyl radicals, tri(C₁-C₆)alkylsilane(C₁-C₆)alkyl radicals, (C₁-C₆)alkoxy(C₁-C₆)alkyl radicals, C₁-C₆ carbamylalkyl radicals, (C₁-C₆)alkylcarboxy(C₁-C₆)alkyl radicals and benzyl radicals, wherein the radicals R₁₆ are borne by a nitrogen atom;
R₁₇ is chosen from C₁-C₆ alkyl radicals, C₁-C₆ monohydroxyalkyl radicals, C₂-C₆ polyhydroxyalkyl radicals, aryl radicals, benzyl radicals, C₁-C₆ aminoalkyl radicals, C₁-C₆ aminoalkyl radicals in which the amine is mono- or disubstituted with at least one radical chosen from (C₁-C₆)alkyl, (C₁-C₆)alkylcarbonyl, amido and (C₁-C₆)alkylsulfonyl radicals; C₁-C₆ carboxyalkyl radicals, C₁-C₆ carbamylalkyl radicals, C₁-C₆ trifluoroalkyl radicals, tri(C₁-C₆)alkylsilane(C₁-C₆)alkyl radicals, C₁-C₆ sulfonamidoalkyl radicals, (C₁-C₆)alkylcarboxy(C₁-C₆)alkyl radicals, (C₁-C₆)-alkylsulfinyl(C₁-C₆)alkyl radicals, (C₁-C₆)alkylsulfonyl(C₁-C₆)alkyl radicals, (C₁-C₆)alkylcarbonyl(C₁-C₆)alkyl radicals, N—(C₁-C₆)alkylcarbamyl(C₁-C₆)alkyl radicals, and N-(C₁-C₆)alkylsulfonamido(C₁-C₆)alkyl radicals,
x is 0 or 1,
when x=0, the linker arm D is attached to the nitrogen atom,
when x=1, the linker arm D is attached to one of the ring members E, G, J, L or M,
Y⁻ is a counterion;
the radicals R₁ and R₂, which may be identical or different, may also be chosen from the onium radical Z of formula:
—X—P(O)(O⁻)OCH₂CH₂N⁺(CH₃)₃
wherein X is chosen from an oxygen atom and a radical —NR₁₈, wherein R₁₈ is chosen from a hydrogen atom, C₁-C₄ alkyl radicals and hydroxyalkyl radicals, the radicals R₁ and R₂, which may be identical or different, may also be chosen from the onium radical Z of formula
—X—C═N⁺(R₁₉R′₁₉)—NR₂₀OR₂₁
wherein X is chosen from an oxygen atom and a radical —NR₂₂, and R₁₉, R′₁₉, R₂₀, R₂₁ and R₂₂, which may be identical or different, are each chosen from a hydrogen atom, C₁-C₄ alkyl radicals and hydroxyalkyl radicals.

8. The composition according to claim 5, wherein the at least one cationic oxidation base is chosen from:
-[1-(4-amino-phenyl)-pyrrolidin-3-yl]-trimethyl-ammonium chloride
-[1-(4-amino-phenyl)-pyrrolidin-3-yl]-dimethyl-tetradecyl-ammonium bromide
-3-[1-(4-amino-phenyl)-pyrrolidin-3-yl]-1-methyl-3H-imidazol-1-ium chloride
-[1-(4-amino-phenyl)-pyrrolidin-3-yl]-(2-hydroxy-ethyl)-dimethyl-ammonium chloride
-[1-(4-amino-phenyl)-pyrrolidin-3-yl]-dimethyl-(3-trimethylsilanyl-propyl)-ammonium chloride
-[1-(4-amino-phenyl)-pyrrolidin-3-yl]-(-trimethylammonium-hexyl)-dimethyl-ammonium dichloride
-[1-(4-amino-phenyl)-pyrrolidin-3-yl]-oxophosphorylcholine
-{2-[1-(4-amino-phenyl)-pyrrolidin-3-yloxy]-ethyl}-trimethyl-ammonium chloride
-1-{2-[1-(4-amino-phenyl)-pyrrolidin-3-yloxy]-ethyl}-1-methyl-pyrrolidinium chloride
-3-{3-[1-(4-amino-phenyl)-pyrrolidin-3-yloxy]-propyl}-1-methyl-3H-imidazol-1-ium chloride
-1-{2-[1-(4-amino-phenyl)-pyrrolidin-3-yloxy]-ethyl}-1-methyl-piperidinium chloride
-3-{3-[1-(5-trimeihylsilanylethyl-4-amino-3-trimethylsilanylethyl-phenyl)-pyrrolidin-3-yloxy]-propyl}-1-methyl-3H-imidazol-1-um chloride
-[1-(4-amino-3-methyl-phenyl)-pyrrolidin-3-yl]-trimethyl-ammonium chloride
-[1-(4-amino-3-methyl-phenyl)-pyrrolidin-3-yl]-dimethyl-tetradecyl-ammonium chloride
-3-[1-(4-amino-3-methyl-phenyl)-pyrrolidin-3-yl]-1-methyl-3H-imidazol-1-ium chloride
-[1-(4-amino-3-methyl-phenyl)-pyrrolidin-3-yl]-(2-hydroxy-ethyl)-dimethyl-ammonium chloride
-[1-(4-amino-3-methyl-phenyl)-pyrrolidin-3-yl]-dimethyl-(3-trimethylsilanyl-propyl ammonium chloride
-[1-(4-amino-3-methyl-phenyl)-pyrrolidin-3-yl]-(-trimethylammonium-hexyl)-dimethyl-ammonium dichloride
-[1-(4-amino-3-methyl-phenyl)-pyrrolidin-3-yl]-oxophosphorylcholine
-{2-[1-(4-amino-3-methyl-phenyl)-pyrrolidin-3-yloxy]-ethyl}-trimethyl-ammonium chloride
-1-{2-[1-(4—amino-3-methyl-phenyl)-pyrrolidin-3-yloxy]-ethyl}-1-methyl-pyrrolidinium chloride
-3-{3-[1-(4-amino-3-methyl-phenyl)-pyrrolidin-3-yloxy]-propyl}-1-methyl-3H-imidazol-1-um chloride
-1-{2-[1-(4-amino-3-methyl-phenyl)-pyrrolidin-3-yloxy]-ethyl}-1-methyl-piperidinium chloride
-[1-(4-amino-3-trimethylsilanylethyl-phenyl)-pyrrolidin-3-yl]-trimethyl-ammonium chloride
-3-[1-(4—amino-3-trimethylsilanylethyl-phenyl)-pyrrolidin-3-yl]-1-methyl-3H-imidazol-1-ium chloride
-3-{3-[1-(4-amino-3-trimethylsilanylethyl-phenyl)-pyrrolidin-3-yloxy]-propyl}-1-methyl-3H-imidazol-1-um chloride
-[1-(5-trimethylsilanylethyl-4-amino-3-trimethylsilanylethyl-phenyl)-pyrrolidin-3-yl]-trimethyl-ammonium chloride
-3-[1-(5-trimethylsilanylethyl-4-amino-3-trimethylsilanylethyl-phenyl)-pyrrolidin-3-yl]-1-methyl-3H-imidazol-1-um chloride
-1′-(4-amino-phenyl)-1-methyl-[1,3′]bipyrrolidinyl-1-ium chloride
-1′-(4-amino-3-methyl-phenyl)-1-methyl-[1,3′]bipyrrolidinyl-1-ium chloride
-3-{[1-(4-amino-phenyl)-pyrrolidin-3-ylcarbamoyl]-methyl}-1-methyl-3H-imidazol-1-ium chloride
-3-{[1-(4-amino-3-methyl-phenyl)-pyrrolidin-3-ylcarbamoyl]-methyl}-1-methyl-3H-imidazol-1-ium chloride
-3-[1-(4-amino-phenyl)-pyrrolidin-3-yl]-1-(3-trimethylsilanyl-propyl)-3H-imidazol-1-ium chloride
-3-[1-(4-amino-phenyl)-pyrrolidin-3-yl]-1-(3-trimethylsilanyl-propyl)-3H-imidazol-1-ium chloride
-[1-(4-amino-phenyl)-pyrrolidin-3-yl]-ethyldimethyl-ammonium chloride
-[1-(4-amino-phenyl)-pyrrolidin-3-yl]-ethyldimethyl-ammonium iodide
-[1-(4-amino-phenyl)-pyrrolidin-3-yl]-propyldimethyl-ammonium iodide
-[1-(4-amino-phenyl)-pyrrolidin-3-yl]-propyldimethyl-ammonium bromide
-[1-(4-amino-phenyl)-pyrrolidin-3-yl]-propyldimethyl-ammonium methosulfate
-[1-(4-amino-phenyl)-pyrrolidin-3-yl]-butyldimethyl-ammonium iodide
-[1-(4-amino-phenyl)-pyrrolidin-3-yl]-pentyldimethyl-ammonium iodide
-[1-(4-amino-phenyl)-pyrrolidin-3-yl]-hexyldimethyl-ammonium iodide
-[1-(4-amino-phenyl)-pyrrolidin-3-yl]-heptyldimethyl-ammonium iodide
-[1-(4-amino-phenyl)-pyrrolidin-3-yl]-octyldimethyl-ammonium iodide
-[1-(4-amino-phenyl)-pyrrolidin-3-yl]-decyldimethyl-ammonium iodide
-[1-(4-amino-phenyl)-pyrrolidin-3-yl]-hexadecyldimethyl-ammonium iodide
-[1-(4-amino-phenyl)-pyrrolidin-3-yl]-hydroxyethyldimethyl-ammonium chloride
-[1-(4-amino-phenyl)-pyrrolidin-3-yl]-hydroxyethyldimethyl-ammonium iodide.

9. The composition according to claim 1, wherein the at least one cationic oxidation base is present in an amount ranging from 0.001% to 10% by weight, relative to the total weight of the composition.

10. The composition according to claim 1, wherein the at least one alcohol oxidase enzyme is chosen from those belonging to the category EC 1.1.3.

11. The composition according to claim 10, wherein the at least one alcohol oxidase enzyme is chosen from primary alcohol oxidases (EC 1.1.3.13), secondary alcohol oxidases (EC 1.1.3.18), long hydrocarbon chain alcohol oxidases (EC 1.1.3.20), polyvinyl alcohol oxidases (EC 1.1.3.30), vanillyl alcohol oxidase (EC 1.1.3.38) and aromatic alcohol oxidases (EC 1.1.3.7).

12. The composition according to claim 11, wherein the at least one alcohol oxidase enzyme is derived from one of the following species: Rhodococcus erythropolis, Pseudomonas pseudoalcaligenes, Aspergillus niger, Kamagataella pastoris, Phanerochaete chrysosporium, Polyporus obtusus, Hansenula polymorpha, Poria contigua, Penicillium simplicissimum, Pleurotus pulmonarius, Pichia pastoris, Pichia methanolica, Pichia angusta, Candida boidinii, Candida albicans, Candida tropicalis, Pinus strobus, Gastropode mollusc, and Manduca sexta.

13. The composition according to claim 12, wherein the at least one alcohol oxidase enzyme is derived from Pichia pastoris.

14. The composition according to claim 1, wherein the at least one alcohol oxidase enzyme is present in an amount ranging from 0.05% to 20% by weight, relative to the total weight of the composition.

15. The composition according to claim 1, wherein the at least one alcohol oxidase enzyme is present in an amount ranging from 10³ U to 10⁵ Upper 100 g of the dye composition.

16. The composition according to claim 1, wherein the at least one substrate for the enzyme is an alcohol chosen from branched and unbranched, saturated and unsaturated, substituted and unsubstituted, primary and secondary alcohols, long hydrocarbon chain alcohols and aromatic alcohols.

17. The composition according to claim 1, wherein the at least one substrate for the enzyme is present in an amount ranging from 0.01% to 60% by weight relative to the total weight of the composition.

18. The composition according to claim 1, further comprising at least one non-cationic oxidation base chosen from para-phenylenediamines, bis(phenyl)alkylenediamines, para-aminophenols, ortho-aminophenols and heterocyclic bases, and the addition salts thereof.

19. The composition according to claim 18, wherein the at least one non-cationic oxidation base is present in an amount ranging from 0.0001% to 20% by weight relative to the total weight of the composition.

20. The composition according to claim 1, further comprising at least one coupler chosen from meta-phenylenediamines, meta-aminophenols, meta-diphenols, naphthalene-based couplers and heterocyclic couplers, and the addition salts thereof.

21. The composition according to claim 20, wherein the at least one coupler is present in an amount ranging from 0.0001% to 20% by weight relative to the total weight of the composition.

22. The composition according to claim 1, further comprising at least one direct dye chosen from natural and cationic direct dyes.

23. A process for dyeing keratin fibers, comprising applying to the keratin fibers at least one composition for a period of time sufficient to develop a desired coloration, wherein the composition comprises, in a medium suitable for dyeing, at least one cationic oxidation base; at least one alcohol oxidase enzyme; and at least one substrate bearing at least one alcohol functional group for said enzyme, wherein said at least one substrate may be totally or partially replaced by at least one dye ingredient in the composition in the case where said at least one dye ingredient in the composition bears at least one alcohol functional group chosen from aliphatic and aromatic alcohol functional groups.

24. The process according to claim 23, wherein the keratin fibers are human hair.

25. The process according to claim 23, wherein the composition is a ready-to-use composition and is stored in anaerobic form, free of oxygen gas.

26. The process according to claim 23, comprising a preliminary operation comprising separately storing, on the one hand, a composition (A) comprising in a medium suitable for dyeing, the at least one cationic oxidation base, and, on the other hand, a composition (B) comprising, in a medium suitable for dyeing, the at least one alcohol oxidase enzyme, wherein at least one of the composition (A) and the composition (B) comprises the at least one substrate for the enzyme and then mixing together the compositions (A) and (B) at the time of use before applying this mixture to the keratin fibers.

27. The process according to claim 26, comprising a preliminary operation comprising separately storing, on the one hand, a composition (A) comprising, in a medium suitable for dyeing, the at least one substrate for the enzyme and the at least one cationic oxidation base and, on the other hand, a composition (B) containing, in a medium that is suitable for dyeing, the at least one alcohol oxidase enzyme, and then mixing together the compositions (A) and (B) at the time of use, before applying this mixture to the keratin fibers.

28. A multi-compartment device, comprising
a first compartment comprising a composition (A) comprising in a medium suitable for dyeing, at least one cationic oxidation base, and
a second compartment comprising a composition (B) comprising, in a medium suitable for dyeing, at least one alcohol oxidase enzyme,
wherein at least one of the composition (A) and the composition (B) comprises at least one substrate bearing at least one alcohol functional group for said enzyme, wherein said at least one substrate may be totally or partially replaced by at least one dye ingredient in the composition in the case where said at least one dye ingredient in the composition bears at least one alcohol functional group chosen from aliphatic and aromatic alcohol functional groups.
